(19) United States
(12) Patent Application Publication
(10) Pub. No.: US 2025/0072902 A1
(43) Pub. Date: Mar. 6, 2025

(54) METHOD FOR HANDLING OR TREATING AN UMBILICAL CORD

(71) Applicant: STEMINOV, Vandoeuvre-Lès-Nancy (FR)

(72) Inventors: Ruben LEJZEROWICZ, Vandoeuvre-Lès-Nancy (FR); Caroline SION, Vandoeuvre-Lès-Nancy (FR)

(73) Assignee: STEMINOV, Vandoeuvre-Lès-Nancy (FR)

(21) Appl. No.: 18/727,886

(22) PCT Filed: Jan. 13, 2023
(86) PCT No.: PCT/FR2023/050046

§ 371 Date: Jul. 10, 2024

(30) Foreign Application Priority Data
Jan. 14, 2022 (FR) FR2200311

(51) Int. Cl. A61B 17/122

(52) U.S. Cl. CPC A61B 17/122

(57) ABSTRACT

Method for handling or treating an umbilical cord using a device, which includes a head having at least one actuator and at least two attachment elements, two working parts, each being capable of engaging by a reversible attachment with an attachment element to form with the head a tool capable of being actuated by the at least one actuator, and a device for moving the head in space. The method involves the steps of attaching a working part onto a first attachment element by moving the head, attaching the other working part onto a second attachment element by moving the head, using the tool on the umbilical cord by actuating the actuator, and separating at least one of the working parts from the corresponding attachment element.

### FIELD OF THE INVENTION

The present invention relates to a method for handling or treating an umbilical cord.

The present invention also relates to a device for handling and treating a biological object. More specifically, the present invention relates to a device comprising elements capable of being actuated with reversible fastenings.

### PRIOR ART

Handling and treating a biological object, in particular an umbilical cord, should be performed in the context of strict work constraints in particular in order to avoid any risk of cross-contamination. This work context is imposed by good manufacturing practices (Good Manufacturing Practices, GMP, in English). Reducing direct handling of the biological object by persons is one amongst the means for limiting the risk of contamination. The use of robotic tools allows reducing human contact with the biological object.

However, the known robotic tools for handling and treating biological objects are not satisfactory.

A known method for handling biological objects in compliance with GMPs consists in sterilising the tools between each handling. However, this method considerably extends the duration of working on the biological objects. Yet, some biological materials, and most particularly those of umbilical cords, should be handled and treated as quickly as possible in order to guarantee the quality of the elements that are extracted therefrom. In addition, sterilisation also involves an increase in the cost of treatment of biological objects, especially in the context of automation of the handling processes.

The invention aims to provide a method that enables handling and treatment of a biological object, and quite particularly an umbilical cord, in a rapid, automatic, repeatable manner, or at a lower cost while reducing the risk of cross-contamination.

### SUMMARY

For this purpose, the present invention relates to a method for handling or treating an umbilical cord using a device comprising:
a head comprising at least one actuator and at least two attachment elements, the at least one actuator being configured to make at least one of the attachment elements rotate about its axis of rotation and/or to make a rectilinear movement at least at one of the attachment elements according to an axis parallel to an axis connecting two attachment elements,
two working parts, each working part being capable of engaging, by means of a reversible attachment, with an attachment element so as to form, with the head, a tool capable of being actuated by the at least one actuator, and
a device for moving the head in the space,
   the method comprising the steps of:
      attaching a working part onto a first attachment element by moving the head,
      attaching the other working part onto a second attachment element by moving the head,
      using the tool on the umbilical cord by actuating the actuator, and
      separating at least one of the useful portions from the corresponding attachment element.

This method allows replacing the working parts rapidly between each handling or treatment in order to reduce the risk of cross-contamination.

Indeed, the movable and complex parts such as the actuator and the attachment elements are placed in the head which does not come into contact with the umbilical cord. Thus, only the working parts are in contact with the umbilical cord. Hence, these parts may consist of parts that are simple and easy to produce in order to be replaced at a lower cost between each handling or treatment in order to reduce the risk of cross-contamination.

The device may be particularly suitable for carrying out operations of moving, cutting, or rinsing the umbilical cord in the context of a method for extracting stem cells from the umbilical cord.

In addition, the head can be placed on any movement device, which allows obtaining a versatile tool that can be easily installed on existing movement devices.

Finally, the reversible fastening is particularly advantageous because it enables an automatic replacement of the working parts of the tool without any human intervention. Hence, this reduces the risk of cross-contamination by contact between a person and the umbilical cord. In addition, the reversible fastening enables a rapid replacement of the working parts. Thus, this enables rapid handling and treatment of umbilical cords in order to guarantee the quality of the elements that are extracted therefrom.

According to another advantageous aspect of the invention, the device comprises at least one fixed attachment element.

This allows handling heavy tools or elements or performing handling operations requiring a greater force.

According to another advantageous aspect of the invention, the device further comprises an independent actuator for each attachment element.

This enables an asynchronous movement of the working parts, which increases the number of degrees of freedom of the tool and therefore increases accuracy during handling or treatment of the umbilical cord. In addition, this enables the use of complex tools such as push syringes.

According to another advantageous aspect of the invention, the at least one actuator is configured to make the attachment elements rotate about their axis of rotation and the axes of rotation of the attachment elements are distinct.

According to another advantageous aspect of the invention, the reversible fastening is magnetic or mechanical.

According to another advantageous aspect of the invention, the reversible fastening is mechanical and comprises a pusher allowing separating the working part from the corresponding attachment element. The device may further comprise a bearing element against which the pusher can be pressed by moving the head in order to release the working part of the corresponding attachment element.

According to another advantageous aspect of the invention, the tool is a clamp and each working part is a jaw of the clamp. Preferably, the jaw of the clamp may comprise several lamellae or teeth spaced apart from one another so as to enable the clamp to grasp a tubular shaped biological object (for example an umbilical cord) while limiting contact with the latter and, at the same time, holding it over its length. Each tooth may also be pierced at its end opposite to the head in order not to damage the umbilical cord which, if its density is low, should have enough free space so that its shape could adapt to the tool that handles it.

The tool may also consist of a liquid sampling or ejection tool, and the working parts may comprise a fastening module of a plunger syringe, and a plunger pusher adapted to move, when the corresponding actuator is actuated, the plunger of a syringe fastened to the fastening module.

The tool may also consist of a cutting tool configured to cut the umbilical cord, and the working parts comprise a blade and a cord pusher.

According to another advantageous aspect of the invention, portions of the working parts intended to be in contact with the umbilical cord comprise a biocompatible material, preferably a polymer material.

The use of a polymer material allows obtaining working parts which are easy and inexpensive to produce. For example, the working parts may be produced by additive manufacturing or 3D printing. Hence, these parts may be replaced at a lower cost between each handling or treatment in order to reduce the risk of cross-contamination.

According to another advantageous aspect of the invention, at least one actuator is controlled by a closed-loop control.

This allows achieving safety in the event of an impact against the working parts. Indeed, when a pressure is exerted on one of the working parts so as to involve a movement of the corresponding attachment element, the actuator could dampen it and then automatically return the position it had before the impact.

In one embodiment, the device for moving the head comprises a three-axis robot arm. The invention can easily adapt to any type of robotic arm or any other device for moving in translation and/or rotation in the space.

In addition, the device may comprise:
at least one sensor configured to locate the umbilical cord, and
a control device configured to control the device for moving the head and the at least one actuator of the head according to the location of the umbilical cord determined by the sensor.

In one embodiment, the sensor is a camera, a colour sensor or a radar.

According to a second aspect, the invention also relates to a device configured for handling or treating a biological object using a liquid sampling or ejection tool capable of being actuated, the device comprising:
a head comprising at least one actuator and at least two attachment elements, the at least one actuator being configured to make at least one of the attachment elements rotate about its axis of rotation and/or to make at least one of the attachment elements perform a rectilinear movement according to an axis parallel to an axis connecting the two attachment elements,
two working parts, each working part being capable of cooperating, by means of a reversible fastening, with an attachment element so as to form, with the head, the tool capable of being actuated by the at least one actuator, and
a device (119) for moving the head in the space, wherein the working parts comprise a fastening module of a plunger syringe, and a plunger pusher adapted to move, when the corresponding actuator is actuated, the plunger of a syringe fastened to the fastening module.

According to a third aspect, the invention also relates to a device configured for handling or treating a biological object by means of a cutting tool capable of being actuated configured to cut the biological object, the device comprising:
a head comprising at least one actuator and at least two attachment elements, the at least one actuator being configured to make at least one of the attachment elements rotate about its axis of rotation and/or to make at least one of the attachment elements perform a rectilinear movement according to an axis parallel to an axis connecting the two attachment elements,
two working parts, each working part being capable of cooperating, by means of a reversible fastening, with an attachment element so as to form, with the head, the tool capable of being actuated by the at least one actuator, and
a device for moving the head in the space, wherein the working parts comprise a blade and a cord pusher.

The present disclosure also provides a device configured for handling or treating a biological object using a tool capable of being actuated, the device comprising:
a head comprising at least one actuator and at least two attachment elements, the at least one actuator being configured to make at least one of the attachment elements rotate about its axis of rotation and/or to make at least one of the attachment elements perform a rectilinear movement according to an axis parallel to an axis connecting the two attachment elements,
two working parts, each working part being capable of cooperating, by means of a reversible fastening, with an attachment element so as to form, with the head, the tool capable of being actuated by the at least one actuator, and
a device for moving the head in the space.

The movable and complex parts such as the actuator and the attachment elements are placed in the head which does not come into contact with the biological object. Thus, only the working parts are in contact with the biological object. Hence, these parts may consist of parts that are simple and easy to produce in order to be replaced at a lower cost between each handling or treatment in order to reduce the risk of cross-contamination.

### Definitions

In the present invention, the terms hereinbelow are defined as follows:

“Biocompatible” relates to the ability of a material not to interfere with or degrade the biological object with which it interacts.

“Good manufacturing practices (GMP)” relates to the principles and guidelines to be complied with for the manufacture of medicines for human and veterinary use. It is one amongst the quality assurance elements, guaranteeing that the products are manufactured and controlled uniformly and according to quality standards suitable for use thereof and specified in the marketing authorisation.

“Closed-loop control” relates to an automatic control of an actuator enabling it to return to a predefined position after having been diverted from this position.

“Reversible fastening” relates to a fastening means which enables attachment and detachment of two parts.

“Tool capable of being actuated” relates to a tool that has several use positions, actuating it making it switch from one position to another or making it carry out a predetermined action.

### BRIEF DESCRIPTION OF THE FIGURES

Other features and advantages of the present invention will appear from the description given hereinbelow, with reference to the appended drawings which illustrate non-limiting embodiments thereof. In the figures:

FIG. 1 shows a top view of a system implemented in a method according to one embodiment of the invention,

FIG. 2 is a view of the system of FIG. 1 according to another viewpoint,

FIG. 3 is a view of the system of FIG. 1 in an embodiment wherein the collector tray is a movable platform

FIG. 4 shows the head of a handling and treatment device of the system in an embodiment according to which the head comprises two actuators which are configured to make the attachment elements rotate about their axis of rotation,

FIG. 5 shows the head of the device in an embodiment according to which the head comprises two actuators which are configured to make the attachment elements perform a rectilinear movement according to an axis parallel to an axis connecting the two attachment elements,

FIG. 6 shows the head of the device in an embodiment according to which the head comprises two fixed attachment elements,

FIG. 7 shows the tool capable of being actuated for gripping a biological object according to two embodiments (narrow clamp on the left-side and wide clamp with teeth on the right-side),

FIG. 8 shows the tool capable of being actuated for gripping a biological object according to two embodiments (curved narrow clamp on the left-side and curved wide clamp on the right-side),

FIG. 9 shows the liquid ejection tool capable of being actuated according to one embodiment and according to three viewpoints,

FIG. 10 shows another example of a working part (hook) that can be used with the device,

FIG. 11 shows the reversible fastening according to the preferred embodiment (a push clip),

FIG. 12 shows the use of the reversible fastening according to the preferred embodiment (a push clip),

FIG. 13 shows the cutting tool according to the preferred embodiment,

FIG. 14 shows an embodiment of the gutter,

FIG. 15 shows an embodiment of the collector tray (movable platform),

FIG. 16 shows the cutting tool (a blade with a rectilinear cutting edge) according to one embodiment,

FIG. 17 shows the cord pusher tool according to one embodiment,

FIG. 18 shows the method according to an embodiment of the invention,

FIG. 19 shows a first step of the method according to an embodiment of the invention,

FIG. 20 shows a second step of the method according to an embodiment of the invention,

FIG. 21 shows a fourth step of the method according to an embodiment of the invention,

FIG. 22 shows a fifth step of the method according to an embodiment of the invention,

FIG. 23 shows the determination of the position of clots according to an embodiment of the invention.

### DETAILED DESCRIPTION

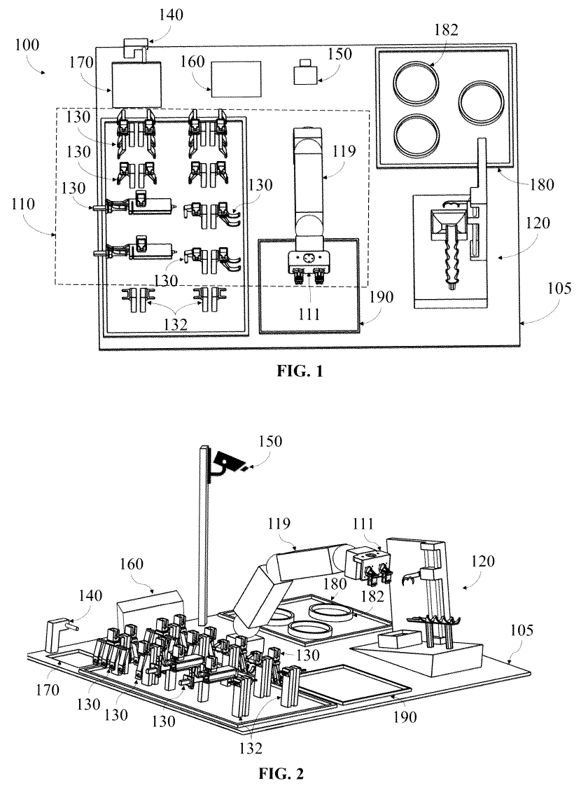

FIGS. 1 to 3 show a system 100 for handling or treating a biological object according to an embodiment of the invention.

The biological object may consist of any type of biological object that could be grasped. Preferably, the biological object is a part of an organ extracted from the body of a living being and in particular an umbilical cord. Thanks to the system 100, this umbilical cord may be handled (for example moved, cut, etc.) or treated (for example by rinsing) in order to extract stem cells therefrom. In the remainder of the description, the system 100 will be described according to application thereof to handling and treatment of an umbilical cord but the present invention could also be used in the context of handling and treating any other biological object that could be grasped such as an organ, preferably with a substantially tubular shape, like, for example, an intestine or an artery.

In particular, the system 100 comprises a device 110 configured for handling or treating a biological object, at least one sensor 150, and a control device 160.

The system 100 may further comprise a cutting tool 120, a handling area 190, and a sample storage area 180 which may include one or more Petri dish(es) 182.

In particular, the device 110 comprises a head 111 and working parts 130.

Advantageously, the head 111 comprises the complex elements of the device. This is advantageous because, during the use of the device 110, the head 111 never comes into contact with the biological object. Thus, there is no risk of cross-contamination between several handlings or treatments, which allows using the same elements composing the head 111 several times. Hence, it is advantageous to include the complex and therefore more expensive element to produce in the head 111. More specifically, as shown in FIGS. 4 to 6, the head 111 comprises at least one actuator 112 and at least two attachment elements 113.

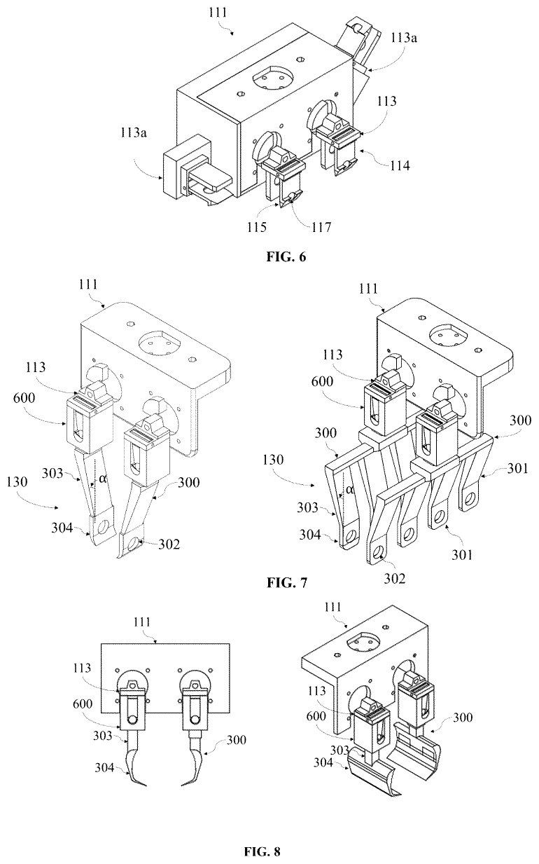

In an embodiment shown in FIGS. 4 and 5, the head 111 comprises two movable attachment elements 113. In an alternative embodiment shown in FIG. 6, the head 111 comprises two movable attachment elements 113 as well as two fixed attachment elements 113a. The fixed attachment elements 113a allow handling heavy tools or elements or performing handlings requiring a very great force compared to the power of the actuators 112.

The actuator 112 is configured to move at least one of the attachment elements 113. In the embodiment illustrated in FIG. 4, the actuator 112 is configured to make two attachment elements 113 rotate about their axis of rotation (Z1, Z2) (the movement of the attachment elements 113 is illustrated by the arrows in the left-side view of FIG. 4). In this embodiment, the actuator 112 is preferably configured to perform a circle-arc rotational movement (limit of rotation). In other words, the actuator 112 is configured to avoid a 360° rotation. This allows perfectly calibrating the actuators 112 by rotation of these up to their limit of rotation. Indeed, when the actuator 112 reaches its limit of rotation, its position can be calibrated and associated, for example, with a 0° angle. Alternatively, in the embodiment illustrated in FIG. 5, the actuator 112 may also be configured to make two attachment elements 113 perform a rectilinear movement (a translation) according to an axis parallel to an axis connecting the two attachment elements 113 (the movement of the attachment elements 113 is illustrated by the arrows in the left-side view of FIG. 5). Still alternatively, the actuator 112 may be configured to make two of the attachment elements 113 rotate about their axis of rotation (Z1, Z2) and to make them translate according to an axis parallel to an axis connecting the two attachment elements 113.

The axes of rotation (Z1, Z2) of the attachment elements 113 may be distinct, i.e. spaced apart from one another. In a preferred embodiment illustrated in FIG. 4, the axes of rotation (Z1, Z2) of the attachment elements 113 are distinct and parallel to one another.

The attachment elements 113a being fixed, they are not actuated by actuators.

The actuator 112 may consist of any element for moving the attachment elements 113 according to the above-described movements. Preferably, the actuators 112 may consist of motors, cylinders, etc. In the case where the actuator 112 is a motor, this may consist of a stepper motor or a servomotor. Preferably, the actuator 112 is a stepper motor which allows setting the position of the motor and therefore of the attachment elements 113 more accurately. Preferably, the stepper motors are controlled by a TMC driver comprising a StallGuard technology enabling the driver to perform an accurate movement without using sensors.

Advantageously, the device 110 comprises one independent actuator 112 for each attachment element 113 as shown in the right-side views of FIGS. 4 and 5. This enables an asynchronous movement of the attachment elements 113 which increases the number of degrees of freedom of the tool and therefore increases accuracy during handling or treatment of the biological object. In addition, this enables the use of complex tools such as a pusher syringe.

In one embodiment, the actuators 112 are fixed, which allows obtaining a tool that cannot be actuated (all of the attachment elements 113 and 113a being fixed).

Advantageously, at least one of the actuators 112 is controlled by closed-loop control. This is advantageous because the closed-loop control allows achieving safety in the event of impact. Indeed, when a pressure is exerted on one of the attachment elements 113 so as to involve a deviation of its position, the actuator 112 will automatically make the attachment element 113 return to the position that it had before the impact.

Preferably, the actuators 112 are mounted in a closed case (hermetically) to avoid any contact with the biological object as shown in FIG. 6. In this particular embodiment, the attachment elements 113 are arranged at the interface of the case in order to link the actuators 112 positioned inside the case to the working parts 130 positioned outside the case. The fixed attachment elements 113a are positioned on the external surface of the case. Preferably, in order to minimise the size of the head 111, the fixed attachment elements 113a are positioned on the sides of the head, i.e. on a different surface of the attachment elements 113.

The attachment elements 113 and 113a enable attachment of the working parts 130 of the tool.

The head 111 is fastened on a device for moving the head 119. This device for moving the head 119 is comprised in the device 110. This movement device 119 allows moving the head 111 in the space in order to reach the different elements of the system 100 or the biological object. The movement device 119 can move over a workspace 105 which is a substantially planar surface whose dimensions are defined beforehand. The dimensions of the working space 105 could at least enable the arrangement of the different working parts 130, of the cutting tool 120, of the handling area 190, of the sample storage area 180 and of a waste discharge area 170. The working area may also comprise a space for the device 110, the sensor 150 and/or the control device 160.

One of the advantages of the present invention is that the head 111 can be mounted on any movement device. This enables the use and the set-up of the device on already existing movement devices. In one embodiment, the movement device 119 comprises a three-axis robot arm.

The movement of the movement device 119 may be controlled automatically by the control device 160 according to the information supplied by the sensor 150, manually remotely or according to a movement pattern recorded beforehand.

The sensor 150 may consist of any type of sensor allowing recording signals in order to determine the position of different objects in the space. For example, the sensor 150 may consist, without limitation, of a colour sensor, a camera or a radar. Advantageously, the colour sensor also allows detecting or confirming the position of blood clots that might be present in the umbilical cord.

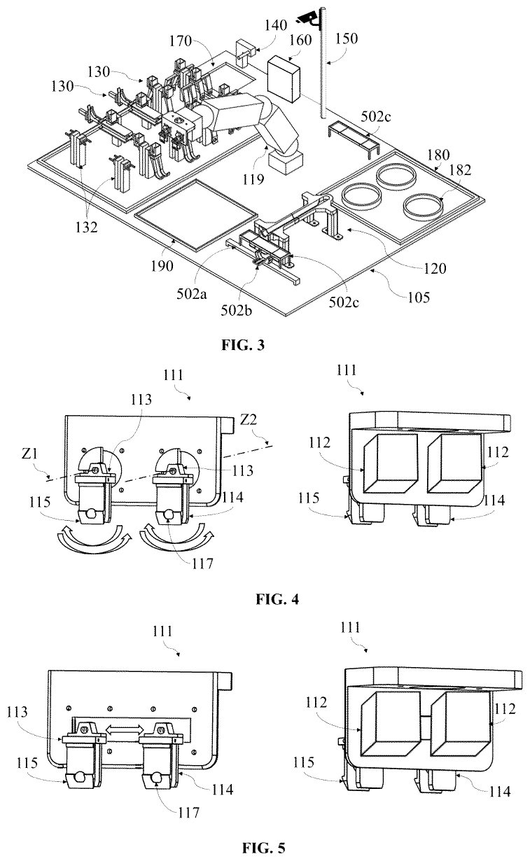

The sensor 150 may be installed on a fixed support (as shown in FIGS. 2 and 3) or on the movement device 119.

In the embodiment wherein the sensor 150 is installed on a fixed support, the sensor 150 is preferably placed at height so as to cover the entire workspace 105.

The sensor 150 may also allow determining the location of the head 111 and of the working parts 130.

Afterwards, the signals recorded by the sensor 150 are sent to the control device 160 in order to be processed therein. The transmission of the signal may be done in a wired or wireless manner.

The control device 160 determines the position in the space and/or the shape of the biological object and, optionally, the position of the tool. These positions are used by the control device 160 in order to generate steering commands which will be executed by the movement device 190 and/or at least one of the actuators 112 and/or the cutting tool 120. The control device 160 may comprise a processor configured to perform the above-described tasks (determination of the position and generation of steering commands). The movement device 190, the actuators 112 and the cutting tool 120 may be controlled by independent controllers.

As described hereinabove, the device 110 also comprises working parts 130. The working parts 130 are elements which will be in contact with the biological object during handling or treatment thereof. Preferably, in order to avoid cross-contamination, the working parts 130 are intended for one single use. This means that they will be discarded, for example, in the waste discharge area 170, after each handling or treatment of the biological object. In other words, the working parts 130 are interchangeable. The device 110 is then compatible with several different working parts.

Advantageously, the working parts 130 consist of simple parts, comprising few mechanical elements, and few or no electronic elements. This is advantageous because this reduces the production cost of these parts intended for one single use.

Advantageously, the working parts 130 are manufactured from a biocompatible material, preferably a polymer material. This is advantageous because the working parts 130 are in contact with the biological object during use of the device 110. Hence, the use of a biocompatible polymer allows using the working parts 130 without interfering or degrading the biological object being handled. In one embodiment, only the portions of the working parts 130 intended to be in contact with the biological material comprise a biocompatible material. For example, the surface of the working parts 130 is treated in order to make it biocompatible.

The working parts 130 are fastened on the head 111 by means of reversible fastenings 600 in order to create a tool capable of being actuated. The attachment 600 is so-called reversible because it enables an easy detachment and a repetitive fastening-detachment cycle. The reversible fastening 600 may be magnetic or mechanical.

In the embodiment wherein the reversible fastening 600 is magnetic, the fastening may consist of an electromagnet allowing producing, when it is powered by an electric current, a magnetic field and fastening, by magnetisation, a working part 130. The detachment of the working part 130 is done by cutting off the current powering the electromagnet.

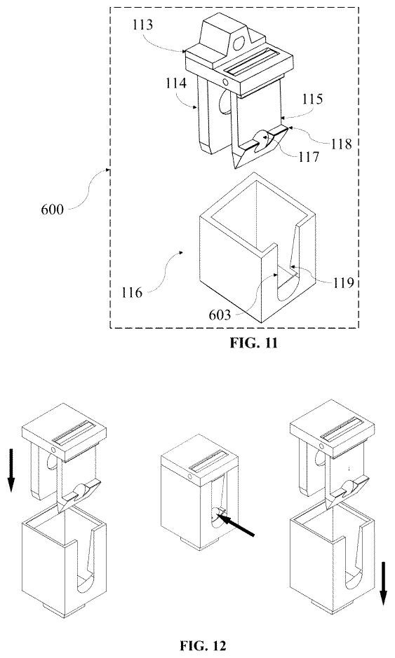

In the embodiment wherein the reversible fastening 600 is mechanical, without limitation, the fastening is one amongst: a clamp, a suction cup, a ball detent or a clip fastener, a screw. Preferably, as shown in FIG. 11, the reversible fastening 600 is a clip fastener equipped with a pusher enabling detachment of the working part 130.

When the reversible fastening 600 is a clip fastener equipped with a pusher (FIG. 11), the fastening comprises at least two portions: a clip (114, 115) and a base 116. In one embodiment, each attachment element (113, 113) comprises a clip (114, 115) and each working part 130 comprises a base 116. In an alternative embodiment, each attachment element (113, 113a) comprises a base 116 and each working part 130 comprises a clip (114, 115). In another alternative embodiment, some of the attachment elements (113 and/or 113b) comprise a base 116 whereas the other attachment elements comprise a clip (114, 115). In this embodiment, each working part 130 of a pair of working parts forming a tool comprises a clip (114, 115) for one of the working parts and abase 116 for the other working part. The clip is composed of a guide 114 (rigid part with a substantially similar size inside the base 116) and a pusher 115 (slightly deformable) comprising a bearing area 117 and movable notches 118. The base 116 has an internal volume able to receive the clip (114, 115) as well as fixed notches 119 configured to enable an easy passage of the movable notches 118 upon insertion of the clip into the base 116 (thereby resulting in the creation of the tool capable of being actuated by closure of the fastening 600 and attachment of the working part 130) thanks to a slight deformation of the pusher 115 (FIG. 12 to the left). Once the movable notches 118 cooperate with the fixed notches 119 (FIG. 12 in the middle), the working part 130 is held on the attachment elements (113, 113a) and cannot be removed by pulling.

In the case where the reversible fastening 600 is a clip fastener equipped with a pusher, the pusher 115 is actuated (thereby resulting in detachment of the working part 130) by a detachment device 140 (FIGS. 1 to 3). The detachment device 140 may comprise a protruding element on which the pusher 115 bears throughout the opening 603 of the base 116 in order to detach the corresponding working part 130 (FIG. 12 in the middle). The protruding element then exerts a pressing force on the bearing area 117 in order to slightly deform the pusher 115. When the movable notches 118 no longer cooperate with the fixed notches 119, the corresponding working part 130 falls by the effect of gravity (FIG. 12 to the right). Preferably, the working part 130 falls into the waste discharge area 170 in order not to contaminate the workspace 105.

In an embodiment shown in FIGS. 7 and 8, the tool capable of being actuated (formed when two working parts 130 are fastened to the head 111 by means of the attachment elements 113) is a clamp and each working part 130 is a jaw 300 the clamp. The jaws 300 are fastened to the attachment elements 113 so that, when the clamp is actuated by the actuators 112, the jaws 300 of the clamp are brought close to one another or away from one another.

Each jaw of the clamp may be composed of one or more teeth 301. Advantageously, each tooth 301 is composed of an upper part 303 and a lower part 304. The upper part 303 is positioned between the attachment element 113 and the lower part 304. This is advantageous because, during use of the device 110, only the lower part 304 comes into contact with the biological object. Hence, the upper part 303 allows defining a safety distance by avoiding any contact between the biological object and the elements of the head 111. Preferably, the upper portion 303 and the lower portion 304 form a non-zero angle α therebetween. Preferably, the angle α is comprised between 5 and 20°. This allows, during closure of the clamp, that only the lower parts 304 come into contact with one another or with the biological object. This also allows correcting the spacing existing between the attachment elements 113 in order to obtain better holding of the biological object in the clamp. Indeed, in the embodiment wherein the attachment elements 113 have distinct and parallel axes of rotation (and therefore the attachment elements 113 have a constant distance therebetween), the angle formed by the upper part 303 and the lower part 304 enables the lower parts 304 to be substantially parallel to one another when the clamp is closed. In the embodiment wherein the attachment elements 113 perform a translation, the angle formed by the upper part 303 and the lower part 304 enables the lower parts 304 to be substantially parallel to one another when the clamp is closed (i.e., when the attachment elements 113 have a minimum distance therebetween).

Advantageously, the lower part 304 is curved as illustrated in FIG. 8. This is advantageous because this particular shape allows catching more easily a biological object placed on a planar surface while avoiding any degradation of the biological object, in particular when the latter has a generally cylindrical geometrical shape like an umbilical cord.

Advantageously, the lower part 304 of each tooth 301 has a hole 302. The hole 302 allows not damaging a biological object which, if its density is low, should have enough free space so that its shape could adapt to the tool that handles it.

In one embodiment, each jaw 300 contains four teeth 301 as shown to the right in FIG. 7. In this embodiment, each tooth 301 is spaced apart by 5 to 30 millimetres, preferably by 15 to 20 millimetres. This allows distributing the weight of the biological object over several contact points in order to minimise the damages caused to the biological object. The use of several teeth allows depositing or grasping an object in a gutter such as that one described hereinafter.

In an alternative embodiment, each jaw 300 contains a wide tooth 301, as shown to the right in FIG. 8. This also allows distributing the weight of the biological object over several contact points in order to minimise the damages caused to the biological object. In this embodiment, the lower part 304 may be made of a slightly deformable material like, for example, silicone or rubber, in order to deform upon contact of the clamp with the workspace and therefore to catch the cord more easily without the jaw breaking up and without degrading the cord.

In an alternative embodiment, each jaw 300 contains a tooth 301 with a small width, as shown to the left in FIGS. 7 and 8, in order to enable a more accurate gripping of small-size biological objects. In one embodiment, the tooth 301 of one of the jaws 300 comprises a saucer intended to collect a part of a biological object during cutting thereof as explained later on. Preferably, the saucer is fastened on the back of the tooth, i.e. towards the outside of the clamp.

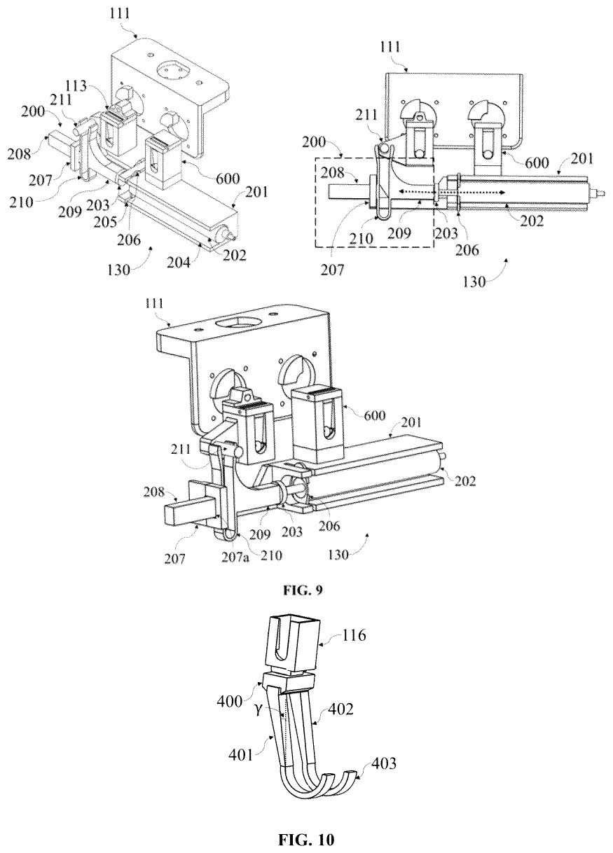

In an embodiment shown in FIG. 9, the tool capable of being actuated (formed when two working parts 130 are fastened to the head 111) is a liquid sampling or ejection tool. One of the working parts 130 is an assembly composed of a fastening module 201 of a plunger syringe and a plunger pusher 200. The other working part 130 is a pin 211 capable of cooperating with the plunger pusher 200.

For example, the fastening module 201 is a single part comprising a body extended by a second part via an arm 207. The body is formed by a first housing 204, for example with a U-like shaped section capable of cooperating with the body 202 of the syringe and a second housing 205 capable of cooperating with the retaining ring (or gripping fins) 206 of the syringe. The body is fastened on one of the attachment elements 113 of the head 111. The arm 207 comprises an opening 207a able to cooperate with the plunger pusher 200. Preferably, the opening is rectangular or square (more generally non-circular) to avoid a rotation of the plunger pusher 200. The centre of the opening 207a of the arm 207 is aligned with the longitudinal axis of the syringe.

The plunger pusher 200 comprises two extensions (208, 209) aligned with the longitudinal axis of the syringe and separated by a transmission structure 210 comprising a U-like shaped guide positioned perpendicular to the two extensions (208, 209). The first extension 209 is the part that comes into contact with the plunger 203 of the syringe in order to exert a pressing or pulling force thereon according to the longitudinal axis of the syringe (illustrated by the dotted arrows in the FIG. 9). The second extension 208 cooperates with the opening 207a of the arm 207 of the fastening module 201. The size of the second extension 208 is selected so that when the plunger pusher 200 completes a pressure movement (the plunger 203 is then completely pushed into the body 202 of the syringe), the second extension 208 is still held in the opening 207a of the arm 207. The arm 207 serves as a stop against which the guide of the transmission structure 210 can bear to limit the stroke of the plunger pusher 200. The transmission structure 210 enables the transmission of the rotational movement of the second attachment element 113 to the plunger pusher 200 by means of the pin 211 fastened on the attachment element 113. The pin 211 can move inside the guide of the guide of the transmission structure 210. This configuration allows creating a translational movement of the plunger pusher 200 parallel to the axis of symmetry of the syringe.

In an embodiment shown in FIG. 10, the tool (formed when two working parts 130 are fastened to the head 111) is a hook 400. For example, the hook comprises a plurality of teeth 401, preferably two teeth 401. Like for the clamp, each tooth may have an upper part 402 and a lower part 403. Like for the clamp, the advantage of this feature is that, during the use of the device 110, only the lower part 403 comes into contact with the biological object or parts of the device having been in contact with the biological object. Hence, the upper part 402 allows defining a safety distance with the head 111. The lower part 403 may have a curved shape, preferably a half-circle shape, whose ends point in the direction of the upper part 402. Preferably, the upper part 402 and the lower part 403 form a non-zero angle γ therebetween. Preferably, the angle γ is comprised between 5 and 15°. Preferably, the hook is a fixed tool (the actuators 112 of the attachment elements 113 are not actuated or the hook is fastened on the fixed attachment elements 113a). Thus, the hook can be used to move elements of the system 100 such as, for example, the gutter 503 described hereinbelow.

In one embodiment, all of the working parts 130 which will be used for handling and treating the biological object are placed beforehand on the workspace 105 on tool supports 132 at a distance such that the movement device 119 can reach it. The working parts 130 are placed on the tool supports 132 so that the inside of the base 116 of the reversible fastening 600 is easily achievable by the clip fastened on the attachment elements (113, 113a).

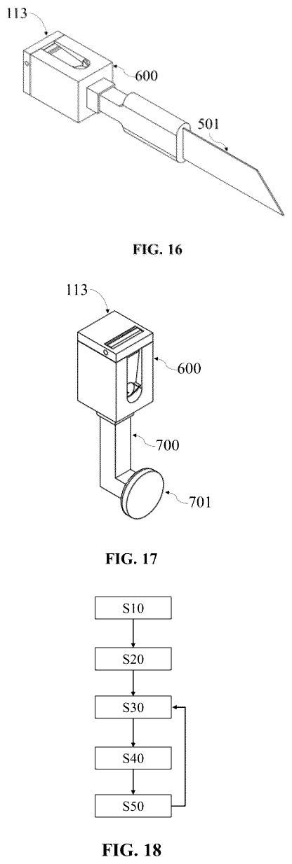

As mentioned hereinabove, the system 100 may also comprise a cutting tool 120 controlled by the control device 160. In the preferred embodiment allowing cutting a umbilical cord, the cutting tool 120 comprises a gutter 503 and a guillotine 507. The guillotine 507 comprises a blade 501 fastened on a movable blade support 506 and a rail 505. The movable blade support 506 can move along the rail 505. In an alternative embodiment, the blade 501 is fastened on a three-axis robot arm. In another alternative embodiment shown in FIG. 16, the blade 501 is a working part 130 intended to be fastened on one amongst the attachment elements (113, 113a) of the head 111 and therefore comprises a base 116.

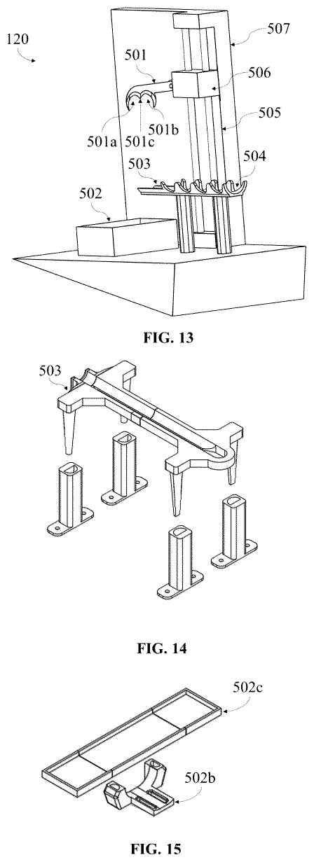

The gutter 503 allows supporting the umbilical cord during cutting of the latter. Preferably, the gutter 503 has a diameter that is slightly larger than the diameter of an umbilical cord which measures on average 1.5 to 2 cm. Preferably, the flanges of the gutter are configured so as to cooperate with the shape of the clamp. For example, when the clamp comprises several teeth 301, the flanges of the gutter comprise recesses 504, each recess 504 being configured to receive one of the teeth 301 of the clamp (FIG. 13). Alternatively, when the clamp contains one single wide tooth 301, the flanges of the gutter have a quite small height fort the clamp to be able to deposit the cord without releasing it by a height that could cause damages to the latter (FIG. 14). Advantageously, the gutter is a movable element (non-fixed) which can therefore be moved. For example, the gutter may be moved by the hook tool 400. In this embodiment, once handling of the biological object is completed, the hook tool is positioned under the gutter in order to lift it in order to remove it from its supports (FIG. 14) and then discard it. Afterwards, a new gutter may be positioned on the supports thanks to transport by the hook tool.

Advantageously, the rail 505 of the guillotine 507 is a double rail comprising two rails parallel to one another. This is advantageous because this allows directing the blade support 506 in a rectilinear manner resulting in a sharp and accurate cut of the umbilical cord.

The blade 501 may consist of any type of blade. Preferably, the blade 501 is a scalpel enabling an accurate cut of the umbilical cord. In the embodiment wherein the blade 501 is mounted on an independent robot arm or on the head 111, the actuator of the robot arm or of the head 111 may perform, if the blade has a substantially rectilinear cutting edge, a reciprocating movement perpendicular to the length of the umbilical cord in order to increase the efficiency and the accuracy of the cut and to avoid crushing of the umbilical cord. Even more advantageously, when the blade 501 is mounted on a rail 505, the blade 501 is a concave double blade, i.e. the cutting edge of the blade 501 is formed by two concave cutting edges (501a, 501b) aligned along the blade 501. The intersection 501c of the two concave cutting edges (501a, 501b) may be located at the middle of the total length of the cutting edge. Advantageously, the intersection 501c of the two concave cutting edges (501a, 501b) is higher than the ends of the cutting edge of the blade so that, when the blade 501 is placed on a planar surface on its cutting edge, only the ends of the cutting edge are in contact with said surface. This is advantageous for cutting an umbilical cord. Indeed, an umbilical cord has a viscous and slippy surface making cutting difficult. During cutting, the intersection 501c of the two concave cutting edges (501a, 501b) is the first contact between the blade 501 and the umbilical cord. The intersection 501c forming a fine tip, this allows holding the umbilical cord in place and thus prevents it from slipping. In addition, the ends of the cutting edge being located lower than the intersection 501c, this enables the blade 501 to wrap the umbilical cord, thereby increasing holding thereof in place as much. The blade 501 is positioned perpendicular to the gutter enabling cutting of segments with a substantially circular section and with a substantially constant thickness. In order to improve cutting of the cord when the blade has a rectilinear cutting edge (rather than double concave), the end of the gutter 503 at which the cut will be performed may comprise a second blade aligned on the blade 501 and whose cutting edge is positioned substantially above the bottom of the gutter 503. In this embodiment, the second blade is a fixed and planar blade. In this manner, the cut is performed at first by the blade 501 and is completed when the blade 501 encounters the second blade.

The cutting tool 120 may further comprise a collector tray 502 positioned under one of the ends of the gutter 503. In one embodiment, the collector tray 502 is fixed (not movable) and may be mounted on springs or not (FIG. 13). Mounting the collector tray on springs is particularly advantageous during recovery of the umbilical cord segment by the device 110. Indeed, this allows imparting a pressure on the bottom of the collector tray in order to completely grasp the segment while avoiding damage to the tray. The internal sides of the collector tray 502 may also be inclined towards the centre of the tray. The latter embodiment is also particularly advantageous during recovery of the umbilical cord segment. Indeed, in this configuration, the absence of a right angle at the bottom of the tray enables the umbilical cord segment to be always completely accessible by the clamp. In an alternative embodiment, the collector tray 502 is a movable platform (FIGS. 3 and 15). The platform comprises a rail 502a on which a support 502b configured to slip laterally along the rail 502a and to receive the tray 502c is positioned. The tray 502c could then be placed on the support 502b, for example by means of a working part provided to this end. After each cut, the tray 502c is moved laterally along the rail 502a over a distance enabling the progressive arrangement of the cut segments along the tray. This lateral movement may be performed by an actuator independent of the head or by a working part which, once attached on the head, exerts a pressure on the tray 502c in the direction of the desired movement. This embodiment is advantageous because the tray can, at the end of cutting of the cord, be placed directly in the storage area without any additional handling of the umbilical cord segments. Advantageously, in the case where the working part 130 is a tooth 301 of the clamp provided with a saucer 305, the cutting tool 120 could be devoid of a collector tray. Indeed, in this embodiment, the head 111 moves before cutting the biological object so that the saucer 305 is positioned under the end of the gutter at which the biological object is cut. Thus, only the tooth 301 should be replaced at the end of handling, which avoids replacing an additional element (the collector tray) between each handling operation. The collector tray 502 is positioned so that the cut umbilical cord segments fall into the collector tray 502 during cutting thereof.

Advantageously, the cutting tool 120 is fastened on an inclined plane. Preferably, the plane is inclined in the longitudinal direction of the gutter 503 so that the end of the gutter 503 at which the cut has a height with respect to the surface of the workspace 105 smaller than the opposite end of the gutter 503. For example, the angle of inclination between the surface of the workspace 105 and the cutting tool 120 is comprised between 2° and 25°. This is advantageous because this enables the cut segments to fall more easily into the collector tray 502 or into the saucer 305. In addition, the inclination of the entire cutting tool 120 allows complying with the perpendicularity between the blade 501 and the gutter 503.

For example, the above-described system 100 allows handling and treating an umbilical cord. Such a method may be useful in the context of preparation of clinical grade mesenchymal stem cells derived from Wharton's jelly, as described in the document WO2018158542.

An example of a method for using the system in this particular case with reference to FIGS. 18 to 22 will be described hereinafter. The umbilical cord is placed beforehand in the handling area 190. Afterwards, the sensor 150 determines the position and optionally the size of the umbilical cord. Optionally, the sensor also determines the position of blood clots in the umbilical cord. Indeed, the cord segments containing these clots cannot be used and should be discarded. In order to determine the position of the clots, the sensor can virtually build a skeleton of the umbilical cord (FIG. 23), i.e. at first defining the centreline 601 of the umbilical cord assuming, for example, that the cord 603 has a tubular shape. Afterwards, sections 602 perpendicular to the centreline 601 are defined. The greater the number of sections 602, the more accurate the position of the clots 604 will be. Afterwards, the position of the clots is defined as the position of the sections in which the clots are located. Alternatively, the position of the clots is determined beforehand and the sensor 150 can then allow validating their position at the time of handling.

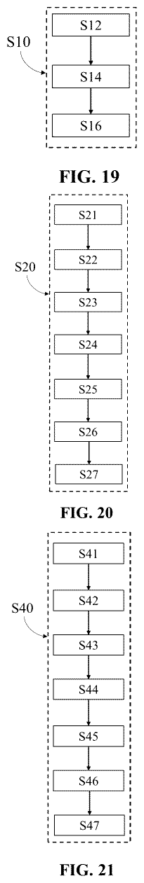

At first, the umbilical cord is treated (step S10, FIGS. 18 and 19). The movement device 119 then allows moving the head 111 to attach, at first, a working part 130 on each of the attachment elements 113 by means of the reversible fastening 600 in order to form the tool capable of being actuated (step S12). For this first step, the tool capable of being actuated is a liquid sampling and ejection tool like that one illustrated in FIG. 9. A syringe prefilled with a rinsing liquid is used. For example, the rinsing liquid may be a phosphate-buffered saline (“phosphate-buffered saline”, PBS). The cord is then treated by external rinsing or by rinsing the umbilical vein by means of the tool capable of being actuated (step S14). For example, rinsing may be performed in a container whose size allows depositing the cord therein without damaging it. In this embodiment, the tray and its contents (the liquid that has been used for rinsing) may be discarded after the treatment. In an alternative embodiment, rinsing may be performed directly on the workspace 105. In this embodiment, the area on which the cord will be rinsed is preferably pierced with one or more hole(s) enabling the discharge of the rinsing liquid. In the context of rinsing of the umbilical vein (whose position can be determined by the sensor), it may be advantageous to keep the umbilical cord in place (for example by inserting it into an element fastened on the workspace) in order to avoid any movement due to the pressure exerted during this rinsing. At the end of the treatment, the working parts 130 are detached (step S16) and may be discarded.

Optionally, after rinsing, the membrane of the umbilical cord can be incised. This incision should have a depth equal to or very slightly larger than the thickness of the membrane in order not to damage the internal cells. For example, the cut may be performed by a rectilinear (rather than double concave) blade. Since the height of the umbilical cord could be not constant over its entire length, the cutting depth should be adjusted along the umbilical cord. For example, this adjustment may be performed by means of the optical sensor measuring, in real-time, the height of the cord at the location of the cut or by means of a force sensor mounted on the blade.

Once the cord is rinsed, the latter can afterwards be cut into segments by the cutting tool 120 (for example that one illustrated in FIG. 13). The umbilical cord is moved from the handling area 190 towards the cutting tool 120 (step S20, FIGS. 18 and 20). This movement step begins by attaching a working part 130 on each of the attachment elements 113 by means of the reversible fastening 600 in order to form the tool capable of being actuated (step S21). For the movement of the umbilical cord, the tool capable of being actuated preferably consists of a long clamp, each jaw 300 of which contains at least two teeth 301, preferably four teeth 301. The movement device 119 then moves the clamp towards the umbilical cord. The head 111 is positioned above the umbilical cord so that the jaws 300 of the clamp are directed parallel to the cord (step S22). The jaws 300 spaced apart by a distance larger than the diameter of the umbilical cord, the head 111 is lowered towards the surface of the workspace 105. The clamp is then closed by means of the actuators 112 so as to grip the umbilical cord without damaging it (step S23). The movement device 119 then moves the tool capable of being actuated and the umbilical cord towards the cutting tool 120 (step S24).

Afterwards, the umbilical cord is deposited in the gutter 503, for example by positioning the teeth 301 of the jaws 300 of the clamp above the recesses 504 of the gutter, then by a vertical movement of the movement device 119 towards the bottom of the recesses 504 (step S25). Preferably, the umbilical cord is positioned so that one of its ends protrudes from the gutter on the side where the cut will be performed. Afterwards, the umbilical cord is deposited by opening the clamp (step S26). At the end of transport, the working parts 130 are detached (step S27). Step S27 is performed in the same manner as step S16.

Afterwards, a segment of the umbilical cord is cut perpendicularly to its length (step S30, FIG. 18) by lowering the blade support 506 along the rail 505 or by a reciprocating movement of the robotic arm or of the head 111. In order to extract the stem cells, the segments preferably have a length comprised between 2 and 3 millimetres. In the case where the collector tray is replaced by a clamp comprising a saucer 305, the use method may comprise, before step S30, a step of attaching at least one working part 130 (a jaw 300 contains a tooth 301 comprising a saucer 305) on at least one of the attachment elements 113 by means of the reversible fastening 600, then a step of moving the head 111 by the movement device 119 in order to position the saucer 305 under the segment to be cut. Preferably, in this embodiment, a working part 130 is fastened on each of the attachment elements 113: the first working part being a jaw 300 contains a tooth 301 comprising a saucer 305 and the second working part being a jaw 300 contains a tooth 301. This allows creating the clamp tool used later on (during step S50).

Thus, the segment cut is moved afterwards towards the sample storage area 180 (step S40, FIGS. 18 and 21). Once the segment has been recovered by the saucer, the movement device 119 then moves the head 111 and the segment towards the sample storage area 180 (step S44) in order to be positioned, for example, above a Petri dish 182 (step S45). Afterwards, the segment is deposited in said Petri dish (step S46) by rotation of the actuator 112 on which the jaw 300 is fastened comprising the saucer 305 or by rotation of the head 111. Once the segment has been recovered by the collector tray 502, a working part 130 is attached on each of the attachment elements 113 by means of the reversible fastening 600 in order to form the tool capable of being actuated (step S41). For moving the segment, the tool capable of being actuated is preferably a narrow clamp, each jaw 300 of which contains a tooth 301. The movement device 119 then moves the tool capable of being actuated towards the segment (step S42). The head 111 is positioned above the collector tray. The jaws 300 being spaced apart by a distance larger than the diameter of the umbilical cord, the head 111 is lowered towards the bottom of the collector tray. The clamp is then closed by means of the actuators 112 so as to grip the segment without damaging it (step S43). The curved shape of the lower part 304 of the tooth 301 is herein advantageous because it allows recovering the segment by the bottom and slightly lifting it from the collector tray 502 before gripping it more firmly. This allows avoiding damaging the segments. The movement device 119 then moves the tool capable of being actuated and the segment towards the sample storage area 180 (step S44) in order to be positioned, for example, over a Petri dish 182 (step S45). Afterwards, the segment is deposited in said Petri dish (step S46). Preferably, the working parts 130 are not detached at the end of transport as long as the cord being handled remains the same. This allows avoiding using these working parts 130 again in the next step. In an alternative embodiment or if cutting of the umbilical cord is completed, the working parts 130 are detached at the end of transport (step S47). Step S47 is then performed in the same manner as steps S16 and S27. Alternatively, instead of moving each segment after each cut, the collector tray may be the movable platform described hereinabove allowing recovering several segments and transporting them together in the storage area.

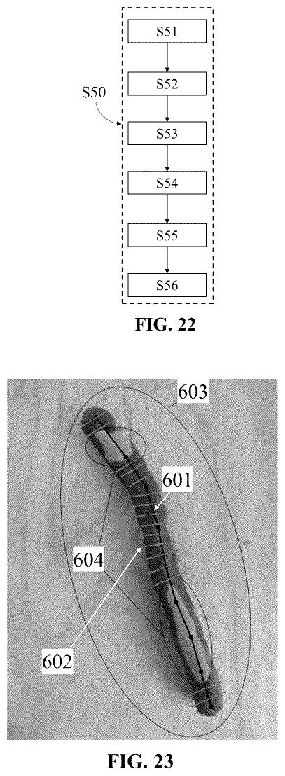

Optionally, the umbilical cord is moved forward afterwards along the gutter in order to cut another segment (step S50, FIGS. 18 and 22). For moving the umbilical cord along the gutter, the tool capable of being actuated is preferably a narrow clamp, each jaw 300 of which contains a tooth 301. Preferably, the working parts 130 of the narrow clamp are the same as in step S40. In the alternative embodiment, a working part 130 is fastened on each of the attachment elements 113 by means of the reversible fastening 600 in order to form the tool capable of being actuated (step S51). The movement device 119 then moves the tool capable of being actuated towards the umbilical cord (step S52). The head 111 is positioned above the umbilical cord so that the jaws 300 of the clamp are directed parallel to the cord and, optionally, the teeth 301 of the clamps are located above the recesses 504. The jaws 300 spaced apart by a distance larger than the diameter of the umbilical cord, the head 111 is lowered towards the gutter. The clamp is then closed by means of the actuators 112 so as to grip the umbilical cord without damaging it (step S53). The movement device 119 then lifts the tool capable of being actuated and the umbilical cord, moves the tool capable of being actuated and the umbilical cord parallel to the gutter in the direction of the collector tray 502 (step S54). Afterwards, the movement device 119 deposits the umbilical cord in the gutter 502 (step S55). Step S55 is then equivalent to step S26. Alternatively, the device 110 comprises, as a working part, a cord pusher 700 configured to push the umbilical cord along the gutter. For example, the cord pusher 700 shown in FIG. 17 comprises a pastille 701, preferably circular, whose size corresponds substantially to the average size of an umbilical cord. For example, this working part may be fastened on a fixed attachment element 113a. The head 111 may then position the working part at the rear of the umbilical cord and impart a translational movement thereon towards the front of the gutter, on the side of the collector tray.

Afterwards, a new segment may be cut (step S30) and transported (step S40), preferably using the same working parts 130 as in this step S50. Preferably, the working parts 130 are not detached at the end of the movement of the cord as long as the handled cord remains the same. This allows avoiding using these working parts 130 again in the next step. In an alternative embodiment, or if cutting of the umbilical cord is completed, the working parts 130 are detached (step S56). Step S56 is performed in the same manner as steps S16, S27 and S47. Alternatively, the blade 506 may be configured to move along the umbilical cord as cuts are performed. The step of moving the umbilical cord forward along the gutter S50 is then suppressed. Optionally, the step of transporting the cut segment S40 is then followed by step S30 of cutting a new segment.

The number of segments to be cut may be determined by the control device 160 according to the total length of the cord which can be estimated by the sensor 150 before cutting. Alternatively, the number of segments may be determined beforehand.

The segments comprising clots are spaced apart. Preferably, the segments comprising clots are discarded in the waste discharge area 170. This step is the same as step S40 of transporting the cut segment towards the sample storage area 180 except that the destination of the segment is the waste discharge area 170 instead of the sample storage area 180.

The present invention also relates to a method for using the above-described device or system. The method comprises the steps of:
attaching a first working part 130 on a first attachment element 113 by moving the head 111,
attaching a second working part 130 on a second attachment element 113 by moving the head 111,
using the tool thus formed on the biological object by actuation of the actuator(s) 112, and
separating at least one working part 130 from the corresponding attachment element 113.

Preferably, this use method is used for each handling or treatment of the biological object in order to avoid cross-contamination.

For example, the working parts 130 used for two consecutive handlings may be the same.

1-10. (canceled)

11. A method for handling or treating an umbilical cord using a device comprising:
a head comprising at least one actuator and at least two attachment elements, the at least one actuator being configured to make at least one of the attachment elements rotate about its axis of rotation and/or to make a rectilinear movement at least at one of the attachment elements according to an axis parallel to an axis connecting two attachment elements,
two working parts, each working part being capable of engaging, by means of a reversible attachment, with an attachment element so as to form, with the head, a tool capable of being actuated by the at least one actuator, and
a device for spatially moving the head,
the method comprising:
attaching a working part onto a first attachment element by moving the head,
attaching the other working part onto a second attachment element by moving the head,
using the tool on the umbilical cord by actuating the actuator, and
separating at least one of the working parts from the corresponding attachment element.

12. The method according to claim 11, wherein the device further comprises an independent actuator for each attachment element.

13. The method according to claim 11, wherein the at least one actuator is configured to make the attachment elements rotate about their axis of rotation and the axes of rotation of the attachment elements are distinct.

14. The method according to claim 11, wherein the reversible fastening is magnetic or mechanical.

15. The method according to claim 14, wherein the reversible fastening is mechanical and comprises a pusher allowing separating the working part from the corresponding attachment element.

16. The method according to claim 11, wherein the tool is a clamp and each working part is a jaw of the clamp.

17. The method according to claim 11, wherein the tool is a liquid sampling or ejection tool, and the working parts comprise a fastening module of a plunger syringe, and a plunger pusher adapted to move, when the corresponding actuator is actuated, the plunger of a syringe fastened to the fastening module.

18. The method according to claim 11, wherein the tool is a cutting tool configured to cut the umbilical cord, and the working parts comprise a blade and a cord pusher.

19. The method according to claim 11, wherein portions of the working parts intended to be in contact with the umbilical cord comprise a biocompatible material.

20. The method according to claim 19, wherein portions of the working parts intended to be in contact with the umbilical cord comprise a polymer material.

21. The method according to claim 11, wherein the device further comprises:
at least one sensor configured to locate the umbilical cord, and
a control device configured to control the device for moving the head and the at least one actuator of the head according to the location of the umbilical cord determined by the sensor.

22. A device configured for handling or treating a biological object using a liquid sampling or ejection tool capable of being actuated, the device comprising:
a head comprising at least one actuator and at least two attachment elements, the at least one actuator being configured to make at least one of the attachment elements rotate about its axis of rotation and/or to make at least one of the attachment elements perform a rectilinear movement according to an axis parallel to an axis connecting the two attachment elements,
two working parts, each working part being capable of cooperating, by means of a reversible fastening, with an attachment element so as to form, with the head, the tool capable of being actuated by the at least one actuator, and
a device for moving the head in the space,
wherein the working parts comprise a fastening module of a plunger syringe, and a plunger pusher adapted to move, when the corresponding actuator is actuated, the plunger of a syringe fastened to the fastening module.

23. A device configured for handling or treating a biological object by means of a cutting tool capable of being actuated configured to cut the biological object, the device comprising:
a head comprising at least one actuator and at least two attachment elements, the at least one actuator being configured to make at least one of the attachment elements rotate about its axis of rotation and/or to make at least one of the attachment elements perform a rectilinear movement according to an axis parallel to an axis connecting the two attachment elements,
two working parts, each working part being capable of cooperating, by means of a reversible fastening, with an attachment element so as to form, with the head, the tool capable of being actuated by the at least one actuator, and
a device for moving the head in the space,
wherein the working parts comprise a blade and a cord pusher.
